(19) United States
(12) Patent Application Publication
(10) Pub. No.: US 2003/0040662 A1
(43) Pub. Date: Feb. 27, 2003

(54) System, method and computer program for monitoring and managing medications

(76) Inventor: Philip Keys, Pittsburgh, PA (US)

Correspondence Address: Debra M. Parrish, Parrish Law Offices, Suite 200, 615 Washington Rd., Pittsburgh, PA, 15228, US

(21) Appl. No.: 10/225,684

(22) Filed: Aug. 22, 2002

Related U.S. Application Data
(60) Provisional application No. 60/314,219, filed on Aug. 22, 2001.

(51) Int. Cl. A61B 5/00

(52) U.S. Cl. 600/300

(57) ABSTRACT

The invention related to a system, method and computer program for monitoring and managing patients within the therapeutic index range for at least one medication which includes receiving, storing and applying at least one set of patient management rules to relating to at least one set of medication-specific data and at least one set of patient-specific data including the therapeutic index and range for such medication, applying said patient management rules to said patient-specific data and said medication-specific data which results in patient management recommendations and storing, displaying and communicating said patient management recommendations. More specifically, the invention discloses a system, method and computer program for managing patients who are taking anticoagulants.

This application claims priority from U.S. Application Ser. No. 60/314,219, filed Aug. 22, 2001, entitled “System, Method and Computer Program for Monitoring and Managing Medications”.

### FIELD OF INVENTION

The present invention relates to a computer-based system and method for monitoring, dosing and managing patients who are prescribed at least one medication that requires continuous monitoring and feedback to achieve a targeted outcome.

### BACKGROUND OF THE INVENTION

Certain conditions, medications and patients require continuous monitoring to achieve the best therapeutic outcome. Unfortunately, health care practitioners often do not have the resources or knowledge to efficiently manage these patients and these patients experience significant adverse events including toxicity and morbidity.

A limited number of computer systems exist for facilitating appropriate drug dosage, assisting with patient management, and supplying information about the pharmacology, adverse effects and drug interactions of medications. See for example U.S Pat. Nos. 5,833,599 and 6,024,699. These existing computer-based systems typically process data relating to use of a drug by a patient and determine the dosage and management of the drug based on patient-specific information. Dosage typically is based on a milligram per patient weight standard that may or may not be modified by predictions or estimates of the patient's pharmcokinetic parameters. However, these computer-based systems are static because they do not provide ongoing analysis and recommendations for managing medications based on the achievement of a defined clinical response. Further, they do not provide a means for integrating clinician input and customization and referring critical cases for clinician review and intervention, which review and intervention may be centralized and remote or on the same site as the patient or treating clinician. Thus, although existing computer systems may be an appropriate tool to manage a variety of conditions or medications, they are ill-suited for medications that have a narrow therapeutic index, particularly those in which failure to remain within the therapeutic index results in significant adverse events.

Similarly, current systems and methods are inappropriate for patients who have severe adverse reactions or events to a prescribed medication, i.e., when the patient has a narrow therapeutic range, or patients who must be maintained within a narrow therapeutic index of drugs having a larger therapeutic index and that may require physician intervention and review.

Further, to the extent that patient management computer systems exist, they do not provide for collection of patient data from multiple sites and the referral of pre-defined cases to a central site for analysis.

Thus, there exists a need for a computer-based system and method for dosing and managing patients who use at least one medication that has a narrow therapeutic index or patients who exhibit a narrow therapeutic range for at least one medication and wherein such system and method provides a means for clinician customization, review and intervention and optionally provides a means for centralizing such intervention and review.

### SUMMARY OF THE INVENTION

The present invention is a computer-based system and process that establishes dosage and patient management parameters using a continuous process that makes dosage and management adjustments based on objective laboratory data measures that reflect specific clinical outcomes and the effect of the medication. This type of system and continuous process is useful to manage medications that have a well-defined clinical response (e.g., a response that can objectively be measured by laboratory testing), but that exhibit substantial variation across the patient population as a function of that well-defined clinical response. Further, such a process and system is necessary to manage patients who have a narrow range of beneficial responses to medication. Successful management of such medications and patients requires a system for continuous monitoring of patient-specific response which responses are well-defined, ongoing adjustment of dosage and other variables known to affect the desired response, and a means of integrating clinician review and intervention for pre-defined criteria wherein such review can be centralized and remote or coincidentally located with the patient or clinician.

An object of the present invention is a means of providing for the continuous monitoring and analysis of a well-defined clinical response to at least one medication.

Another object of the present invention is to determine if an adjustment in dosage or other influencing variable is required to maintain the patient's clinical response within a therapeutic range.

A third object of the present invention is to provide a clinician with at least one recommendation for adjusting variable(s) in a manner that will maintain a patient's clinical response within the desired therapeutic range.

A fourth object of the present invention is to deliver information to and receive information from a clinician based on pre-determined criteria for the purpose of refining or determining recommendations for adjusting patient specific variables(s) in a manner that will maintain the clinical response within the therapeutic range.

A fifth object of the present invention is to provide patient and drug-specific dosage and management recommendations in a printed format which may be distributed to a patient or clinical practitioner.

A sixth object of the present invention is to distribute and receive patient and laboratory information to and from a clinical site or patient site via the internet to a central repository for dosage and management determinations and for archiving historical patient-specific information.

A seventh object of the present invention is to receive input from a clinical or patient site that defines a targeted clinical outcome for a given patient.

An eighth object of the present invention is to deliver information and recommendations to a clinical site or a patient site via the internet.

A ninth object of the present invention is to maintain scheduling and demographic information on each patient to assist in arranging for and monitoring compliance to orders for measurements of clinical response.

Another object of the invention is to provide an algorithm that compares a well-defined clinical response with a standardized acceptable clinical response and to make recommendations based on such a comparison.

Upon reading the following detailed description, other objects will be readily apparent to one skilled in the art.

### BRIEF DESCRIPTION OF THE DRAWINGS

FIG. 1 depicts a preferred embodiment in which information is exchanged between a clinical site, a clinician and a computer system of the present invention via the Internet.

FIG. 2 depicts a preferred embodiment of an information input screen of the present invention that can be used by a clinical site to input information into the system.

FIG. 3 depicts an illustration of a preferred embodiment of the output produced by the present invention.

### DETAILED DESCRIPTION

The present invention is a system and process that uses computer software to compute recommended dosages and patient management decisions based on variables input into the system and that preferably provides a means for integrating physician input and customization and referring cases that meet defined criteria to a physician for review which review may be centralized, remote and/or coincidentally located with the patient. The computer software of the present invention has a means for accepting, storing and conducting analyses with target medication information including commercially available dosages, contraindications, influencing variables, the therapeutic index range, the pharmacokinetic profile and recommended monitoring frequency for at least one drug and preferably for a plurality of drugs.

The software of the present invention also includes a means to accept, store and manipulate patient-specific information and other influencing variables such as other medications, diet, exercise and alcohol consumption into a set of decision rules that appropriately weights such factors in making medication and patient specific recommendations. The computer program of the present invention incorporates such information in making dosage and patient management recommendations.

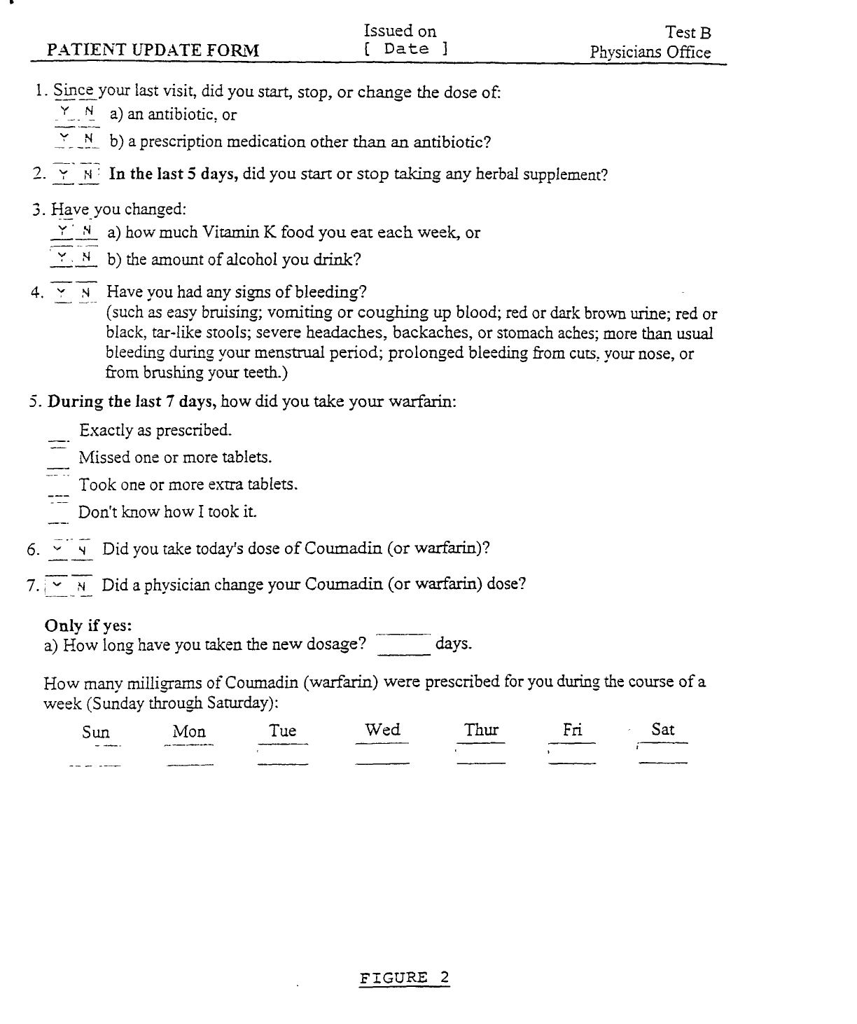

In one embodiment of the invention, patient-specific information is entered to establish a baseline on specified criteria such as the patient's medical history and the targeted clinical response range. Then, on an on-demand basis, information about a patient's current status and laboratory results can be entered into the software of the present invention for analysis and generation of dosage and patient management recommendation. In one embodiment of the invention, the software of the present invention poses a series of questions that solicit required or desired patient-specific information about confounding factors that are known to impact a patient's response to the subject medication (See FIG. 2). For example, the software of the present invention may prompt a patient or clinician to provide information that relates to confounding factors for a therapeutic index such as changes in a patient's diet, alcohol consumption, herbal supplements, and drug-drug interactions.

The present invention uses computer software to (1) provide recommendations for monitoring clinical responses, including the frequency of monitoring events and (2) provide recommendations to adjust the dosage and other influencing variables of the subject medication and other medications. In one preferred embodiment, software is used to securely transmit information from a clinical site, across a communication means, e.g., the Internet, to a server hosting the software of the present invention.

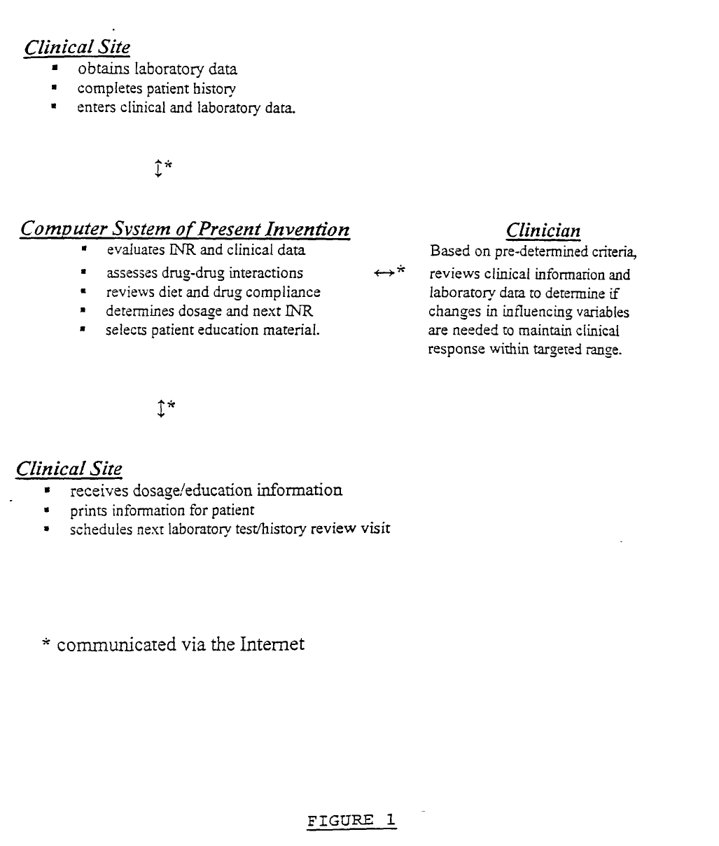

In one embodiment of the invention, patient-specific information is input at a clinical site and transmitted across the Internet to a central repository hosting the software of the present invention. In another embodiment, consistent with home testing which provides the requisite data on a defined clinical response, a patient inputs the patient-specific information. After the software of the present invention analyses the input data and determines dosage patient management recommendations, the recommendations are transmitted back across the Internet to the site at which such recommendations can be implemented. FIG. 1 depicts the flow of medication management information between a clinical site, a clinician, and a server hosting the software of the present invention.

In that preferred embodiment, the clinical information and laboratory results are transmitted across the Internet to a computer system of the present invention hosting the software of the present invention, where such clinical information and laboratory results are analyzed to determine if changes in influencing variables (including dosage) are necessary to maintain the clinical response to the subject medication(s) within the targeted clinical response range(s). Based on pre-determined selection criteria, including alert criteria, the present invention's computer system either determines the appropriate monitoring parameters and changes in dosage and other influencing variables, or forwards the clinical information and laboratory results to a clinician, which clinician may be located at a central, remote or coincident site for such determination.

In a preferred embodiment, a specifically formatted computer screen can be used to present data to a clinician and indicate recommendations suggested by the invention's computer system which includes an algorithm with standardized therapeutic ranges and/or critical observations. A clinician can analyze the information presented in this format, i.e., raw data and recommendations, determine the appropriate monitoring parameters, make changes to variable(s), and forward the recommendations to the present invention's computer server for subsequent application, i.e., a clinician can change the rules for analysis. The present invention's computer system and process allows a clinician to choose and modify the criteria under which clinical information and laboratory are forwarded to a clinician site for further analysis and intervention.

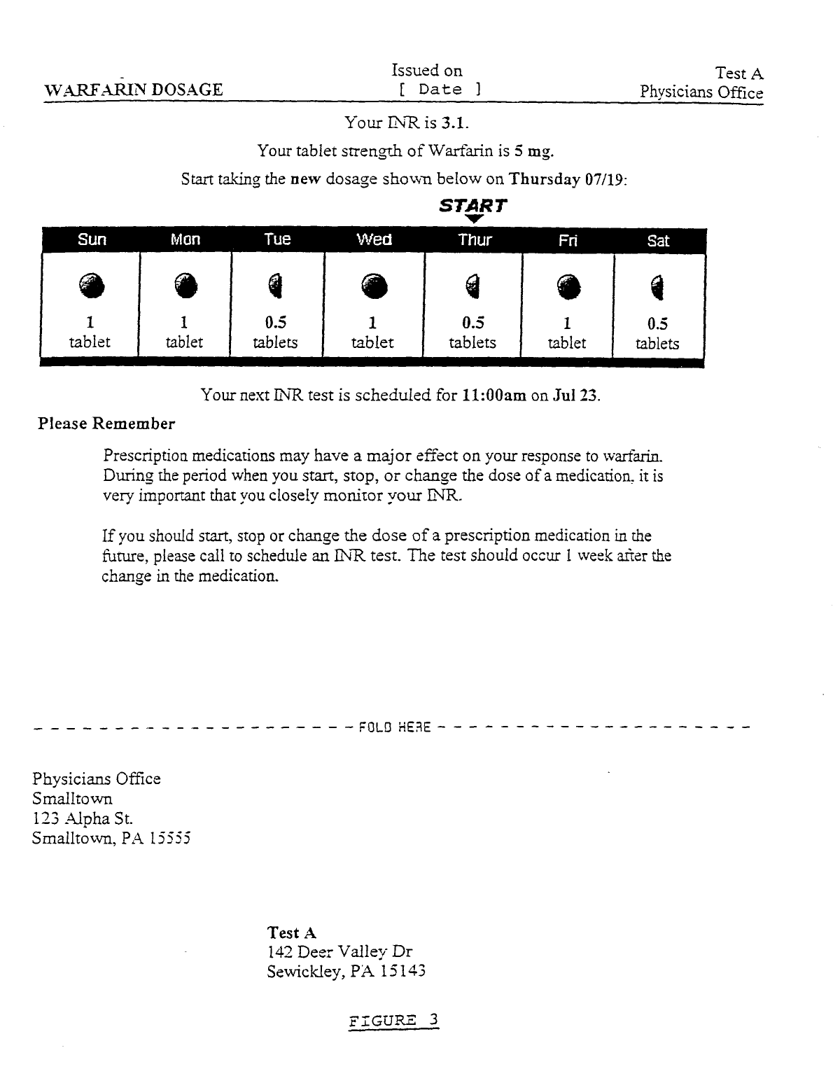

Recommendations about a specific medication's drug dosage and management variables, whether generated by the present invention's computer or by a clinician, are input and stored into the computer system of the present invention. In one embodiment, the information is communicated across the Internet to a clinical site from a distant location hosting the computer software of the present invention. The recommendations generated by the present invention can be presented and printed in a plurality of formats which can be used by the physician or the patient to educate the patient or physician on dosage and monitoring recommendations. See FIG. 3. Further, educational information and recommendations that are designed to alter other influencing variables (e.g., diet) can be presented and printed for use by patients based on their specific needs. Further, recommendations for managing clinical responses outside of the targeted clinical response range (e.g., administration of Vitamin K if the present invention is used to manage anticoagulants) can be presented and printed for use by clinical practitioners at a clinical site.

One embodiment of the present invention includes a scheduling component that facilitates monitoring in compliance with monitoring recommendations. Users at a clinical or scheduling site can use the present invention to schedule follow-up measurements of the clinical response based on recommendations provided by the present invention or a patient's clinician. Users at a clinical or scheduling site can also use the present invention's scheduling component to reschedule patients. The scheduling component of the present invention optionally monitors missed appointments for follow-up measurements of the clinical response, alerts clinical personnel of the missed appointments, and can be used to generate correspondence regarding missed appointments. Studies have shown that such alerts maximize patient compliance.

### EXAMPLE 1

### Anticoagulant Management

One medication that has a narrow therapeutic index and requires continuous monitoring and feedback to achieve a targeted outcome is the anticoagulant warfarin. Warfarin is used to prevent the formation of blood clots in patients with atrial fibrillation, valve replacements, stroke, myocardial infarction, heart failure, deep vein thrombosis, and pulmonary embolism.

Warfarin, like many anticoagulants, is a difficult drug to monitor because it requires frequent blood tests and dosage adjustments to maintain an acceptable therapeutic response. Too little drug is ineffective in preventing the targeted thromboembolic event and too much can result in serious bleeding complications. An analysis of reports in the medical literature demonstrate that in routine medical care the incidence of thromboembolic events averages 16.2% and the incidence of major bleeds averages 10.9%. The Agency for Health Care Policy and Research (“AHCPR”) recently announced that increasing the use of warfarin in patients with atrial fibrillation could prevent as many as 40,000 strokes annually and save nearly $600 million a year in health care costs. AHCPR noted that only 35% of these patients actually receive the drug and only half of them receive the optimal dosage. The primary barrier to increased usage is often identified as the time-consuming and complex process of intensive patient monitoring. Monitoring warfarin therapy through routine medical care is typically fragmented, and the percentage of patients stabilized within the therapeutic range is often less than optimal. Recent studies have shown that more consistent and systematic monitoring and adjustment of dosage significantly improves clinical outcomes and reduces the incidence of complications.

A number of computer programs exist to facilitate management of patients on warfarin. Such computer programs include Coumacare, CoagClinic, DoseResponse, Dawn AC and CleverClog. Each of these programs is static, do not provide a means for integrating clinician review and intervention for a pre-defined set of criteria wherein such clinician is either proximally or distally located to either the patient or data collection site, and do not provide analysis of patient laboratory results against the therapeutic index for warfarin, do not provide a means for inputting variables that affect the patient's adherence to the therapeutic index range, and do not provide recommendations for patient management. For example, Coumacare is an access database management system that allows a user to enter laboratory values, clinical backgrounds, compliance information and outcomes information. Coumacare, however, simply provides a means for storing the laboratory information and does not provide a means for recommending patient management actions, does not provide information regarding suggested therapeutic ranges, does not solicit information from the patient or physician and does not provide a means for integrating clinician input. Similarly, CoagClinic allows a user to store information about patients on warfarin and provides a means for depicting a set of recommended guidelines, but does not provide analysis of patient data against such guidelines, does not make recommendations regarding patient management.

The software of the present invention can support the consistent and systematic evaluation, monitoring and dosage adjustment of warfarin patients. One of the factors that the clinicians may use in managing patients on warfarin, in addition to numerous other variables, are the results of a specific laboratory test which report the levels of warfarin in the international normalized ratio (“INR”) and comparing such results to generally accepted recommended ranges.

When the present invention is first applied to manage a patient, baseline patient-specific data, which data has been determined to be necessary or desirable for proper patient management, is entered into and accepted by the software of the present invention's computer system. In the present example, such patient-specific information will include at least one member, and preferably a plurality of members, from the following group: demographic data, indication for therapy, targeted therapeutic range, relevant past medical history, current warfarin dosage, INR laboratory value(s), at least one clinician's assessment of patient compliance, influencing variables (e.g., alcohol consumption, use of vitamins), and indicia of bleeding. In one embodiment of the present invention, such information is transmitted from a remote clinical or record-keeping site to a server of the present invention's computer system by a communication means which means could include the Internet.

The computer system of the present invention conducts an analysis of the factor(s) input into the present invention and compares the input variables, and combinations thereof, with generally accepted guidelines and variables that have been input into the system as appropriate patient management parameters. Such parameter preferences may be set by the physician using the present invention or can be based on generally accepted guidelines. For example, the American College of Chest Physicians has issued guidelines on warfarin management which may be input as a set of recommendation rules to be applied to the input variables. Alternatively, an algorithm provided by pharmaceutical manufacturers may be input as a set of analysis rules. The analysis may include evaluation of clinical data and laboratory results; assessment of drug interactions, diet and adverse events; and drug compliance and patient education needs.

These recommendations may be forwarded for immediate clinical application, i.e., patient management, or may be presented to a clinician, along with clinician-selected input variables for a clinician's review, analysis and possible adjustment and input. Such clinician may be co-located at the patient management site or the data input site or may be located at a site remote from either or both such sites. Such recommendations may include dosage adjustment, follow-up INR, identification of patients requiring triage, patient education needs, administration of vitamin K and recommended date for the patient's next visit.

After analysis and recommendation by the present invention, and optionally with review, adjustment and recommendation from a clinician, the recommendations are presented to a clinical site for implementation. In one embodiment of the invention, the recommendations for patient management are sent from a remote server site of the present invention to the clinical site via the Internet. In yet a more preferred embodiment of the invention, the present invention generates appropriate patient-specific educational material which information is communicated from a remote site to a clinical site via the Internet. In one preferred embodiment, specific patient management recommendations and/or educational material may be stored, printed or distributed, electronically or otherwise, to a patient's medical record and/or provided to the patient for his or her education. A clinician can select and designate the variable(s) or combinations of variables which mandate clinician intervention or which accept recommendations generated by the present invention without clinician intervention, for such recommendations to be forwarded for clinical implementation.

Based on subsequent patient encounters, updated data may be input into the present invention. With respect to warfarin, such data may include one or more of the following: assessments of compliance, evidence of bleeding, changes in current dosage, the INR laboratory value, and influencing variables including drug interactions, herbal supplements, and changes in ingestion of alcohol and foods high in vitamin K or vitamin K itself.

In a preferred embodiment encompassing a scheduling module, during patient encounters or scheduling queries, recommendations for monitoring INR's can be accompanied by a scheduling screen that allows the clinical or scheduling site to determine the day (and optionally the time) of the next recommended INR test, schedule that day (and optionally the time), and communicate the day (and optionally the time) in conjunction with or independent of the specific management recommendations and/or educational material that is stored, printed and or distributed to the patient and medical record.

The present invention also provides for medical management alerts to accompany recommendations for monitoring and dosage adjustment. Such alerts can be generated based on patient management parameters entered into the invention. Such medical management alerts can be programmed to automatically refer the information for clinician review, intervention and potential adjustment. With respect to warfarin, alerts may be programmed when the input information includes evidence of any of the following: bleeding, an INR greater that 20, when oral administration of Vitamin K is recommended, or the combination of either a potentially interacting medication or an herbal supplement and an elevated INR.

Thus, the present invention, can be used to manage baseline and updated patient-specific information; apply patient-management, drug specific rules based on accepted or physician designated standards for management recommendations; generate and distribute educational materials and management recommendations; schedule a patient's INR test, monitor and correspond with the patient about missed appointments, view the patient's warfarin management history, reprint recommendations and educational material, and generate reports. Such reports may include the patient's warfarin management history, the patient's quality assurance record including percentage of time in the therapeutic range, and administrative reports.

Clinicians using the method of the present invention will input patient specific data, have such data compared against recommended guidelines, provide a manual or automated means for clinician intervention, review and adjustment.

What is claimed is:

1. A system for monitoring and managing patients within the therapeutic index range for at least one medication comprising:
A computer system having a means of receiving, storing and applying at least one set of patient management rules to relating to at least one set of medication-specific data and at least one set of patient-specific data;
A means of receiving and storing at least one set of medication-specific data into said computer system about at least one medication wherein said medication-specific data includes a therapeutic index range for said medication;
A means of receiving and storing said set of patient-specific data into said computer system about a patient using said medication wherein said patient-specific data includes data relating to said therapeutic index;
Applying said patient management rules to said patient-specific data and said medication-specific data which results in patient management recommendations;
A means of storing, displaying and communicating said patient management recommendations.

2. A system according to claim 1 further comprising managing a patient based on said patient management recommendations.

3. A system according to claim 1 wherein said computer system includes a means of communicating said patient-specific data and medication specific data from at least one remote location to a central location for analysis and/or clinician evaluation.

4. A system according to claim 3 wherein said communication means comprises the internet.

5. A system according to claim 1 wherein said patient management rules identify inputs or analyses that require clinician review and such inputs or analyses are immediately communicated to a clinician.

6. A system according to claim 1 wherein a clinician can modify said patient management rules.

7. A system according to claim 1 wherein said medication-specific data further includes at least one element from the group comprising: commercially available dosages, contraindications, influencing variables, pharmacology, adverse effects, pharmacokinetic parameters and recommended monitoring frequency.

8. A system according to claim 1 wherein said patient-specific data further includes at least on element from the group comprising medical status, diet, exercise, alcohol consumption, patient medication compliance, patient pharmacokinetic parameters, use of supplements or vitamins, weight and age.

9. A system of claim 1 wherein the computer system further comprises a scheduling module that provides a means for scheduling patient visits consistent with patient management recommendations.

10. A system of claim 1 that further comprises a patient education module which includes a means of receiving, storing and transmitting patient education material, selection of said patient education material based on said patient management recommendations, and a means for printing said patient educational material.

11. A method for monitoring and managing patients within the therapeutic index range for at least one medication comprising the steps of:
inputting into a computer system at least one set of patient management rules to relating to at least one set of medication-specific data and at least one set of patient-specific data wherein said computer system has a means of receiving, storing and applying;
inputting and storing at least one set of medication-specific data into said computer system about at least one medication wherein said medication-specific data includes a therapeutic index range for said medication;
inputting and storing said set of patient-specific data into said computer system about a patient using said medication wherein said patient-specific data includes data relating to said therapeutic index;
analyzing said patient-specific data and said medication-specific data in light of said patient management rules which results in patient management recommendations; and
Storing, displaying and communicating said patient management recommendations.

12. A method according to claim 11 further comprising the step of managing a patient based on said patient management recommendations.

13. A method according to claim 11 further comprising communicating said patient-specific data and medication specific data from at least one remote location to a central location for analysis and/or clinician evaluation.

14. A method according to claim 13 wherein said communication occurs via the internet.

15. A method according to claim 11 further comprising the step of communicating to a physician inputs or analyses that require clinician review based on and such inputs or analyses.

16. A method of claim 11 further comprising the step of scheduling a patient for visits consistent with said patient management recommendations.

17. A method of claim 11 further comprising the steps of inputting patient education materials and printing said patient educational materials.

18. A computer program for monitoring and managing patients within the therapeutic index range for at least one medication comprising:
computer readable code for receiving, storing and applying at least one set of patient management rules to relating to at least one set of medication-specific data and at least one set of patient-specific data;
computer readable code for receiving and storing at least one set of medication-specific data into said computer system about at least one medication wherein said medication-specific data includes a therapeutic index range for said medication;
computer readable code for receiving and storing said set of patient-specific data about a patient using said medication wherein said patient-specific data includes data relating to said therapeutic index;
computer readable code for applying said patient management rules to said patient-specific data and said medication-specific data which results in patient management recommendations;
computer readable code for storing, displaying and communicating said patient management recommendations.

19. A computer program according to claim 18 further comprising computer readable code for communicating said patient-specific data and medication specific data from at least one remote location to a central location for analysis and/or clinician evaluation.

20. A computer program according to claim 19 further comprising computer readable code for transmitting data and analysis across the internet.

21. A computer program according to claim 18 further comprising computer readable code that analyzes said patient management rules, patient management recommendations, and inputs and identifies inputs, analyses or recommendations that require clinician review and communicates said inputs, analyses or recommendations to a clinician.

22. A computer program according to claim 18 wherein a clinician can modify said patient management rules.

23. A computer program according to claim 18 wherein said medication-specific data further includes at least one element from the group comprising:
commercially available dosages, contraindications, influencing variables, pharmacology, adverse effects, pharmacokinetic parameters and recommended monitoring frequency.

24. A computer program according to claim 18 wherein said patient-specific data further includes at least on element from the group comprising medical status, diet, exercise, alcohol consumption, patient medication compliance, patient pharmacokinetic parameters, use of supplements or vitamins, weight and age.

25. A computer program of claim 18 further comprising a computer readable code for a scheduling module that provides a means for scheduling patient visits consistent with patient management recommendations.

26. A computer program of claim 18 that further comprises computer readable code that provides a means of receiving, storing and transmitting patient education material, selecting said patient education material based on said patient management recommendations, and printing said patient educational material.

27. A computer program of claim 18 further comprising an algorithm for computing recommended dosages for at least one medication.

28. A system for monitoring and managing patients within the therapeutic index range for anticoagulants comprising:
A computer system having a means of receiving, storing and applying at least one set of patient management rules to relating to at least one anticoagulant and at least one set of patient-specific data;
A means of receiving and storing data relating to at least one anticoagulant into said computer system which includes a therapeutic index range for said anticoagulant;
A means of receiving and storing said set of patient-specific data into said computer system about a patient using said anticoagulant wherein said patient-specific data including the patient's international normalized ratio for said anticoagulant;
Comparing said international normalized ratio with recommended ranges;
Applying said patient management rules to said patient-specific data and said anticoagulant data which results in patient management recommendations;
A means of storing, displaying and communicating said patient management recommendations.

29. A system according to claim 28 further comprising managing a patient based on said patient management recommendations.

30. A system according to claim 28 wherein said computer system includes a means of communicating said patient-specific data and anticoagulant data from at least one remote location to a central location for analysis and/or clinician evaluation.

31. A system according to claim 30 wherein said communication means comprises the internet.

32. A system according to claim 28 wherein said patient management rules identify inputs or analyses that require clinician review and such inputs or analyses are immediately communicated to a clinician.

33. A system according to claim 28 wherein a clinician can modify said patient management rules.

34. A system according to claim 28 wherein said anticoagulant data further includes at least one element from the group comprising: commercially available dosages, contraindications, influencing variables, accepted recommended dosage ranges, pharmacology, adverse effects, pharmacokinetic parameters and recommended monitoring frequency.

35. A system according to claim 28 wherein said patient-specific data further includes at least on element from the group comprising: medical status, diet, exercise, alcohol consumption, assessments of patient medication compliance, patient pharmacokinetic parameters, use of herbal supplements or vitamins, consumption of foods high in vitamin K, consumption of vitamin K, evidence of bleeding, weight and age.

36. A system of claim 28 wherein the computer system further comprises a scheduling module that provides a means for scheduling patient visits consistent with patient management recommendations.

37. A system of claim 28 that further comprises a patient education module which includes a means of receiving, storing and transmitting patient education material relating to anticoagulants, selection of said patient education material based on said patient management recommendations, and a means for printing said patient educational material.

38. A method for monitoring and managing patients within the therapeutic index range for at least one anticoagulant comprising the steps of:
inputting into a computer system at least one set of patient management rules to relating to at least one anticoagulant and at least one set of patient-specific data wherein said computer system has a means of receiving, storing and manipulating said rules and patient-specific data;
inputting and storing data for at least one anticoagulant into said computer system about at least one medication wherein said medication-specific data includes a therapeutic index range for said anticoagulant and said computer systems has a means of receiving, storing and manipulating said anticoagulant data;
inputting and storing said set of patient-specific data into said computer system about a patient using said anticoagulant wherein said patient-specific data includes data relating to the patient's international normalized ratio;
analyzing said patient-specific data and said anticoagulant data in light of said patient management rules which results in patient management recommendations; and
Storing, displaying and communicating said patient management recommendations.

39. A method according to claim 38 further comprising the step of managing a patient based on said patient management recommendations.

40. A method according to claim 38 further comprising communicating said patient-specific data and anticoagulant data from at least one remote location to a central location for analysis and/or clinician evaluation.

41. A method according to claim 38 wherein said communication occurs via the internet.

42. A method according to claim 38 further comprising the step of communicating to a physician inputs or analyses that require clinician review based on and such inputs or analyses which includes at least one member of the group comprising:
evidence of bleeding, medication interaction, an international normalized ratio in excess of 20, use of an herbal supplement, when administration of vitamin K is recommended, an elevated international normalized ratio or physician defined criteria.

43. A method of claim 38 further comprising the step of scheduling a patient for visits consistent with said patient management recommendations.

44. A method of claim 38 further comprising the steps of inputting patient education materials and printing said patient educational materials.

45. A computer program for monitoring and managing patients within the therapeutic index range for an anticoagulant comprising:
computer readable code for receiving, storing and applying at least one set of patient management rules to relating to at least one anticoagulant and at least one set of patient-specific data;
computer readable code for receiving and storing data into said computer system about at least one anticoagulant wherein said anticoagulant data includes a therapeutic index range for said anticoagulant;
computer readable code for receiving and storing said set of patient-specific data about a patient using said anticoagulant wherein said patient-specific data includes the patient's international normalized ratio;
computer readable code for applying said patient management rules to said patient-specific data and said anticoagulant data which results in patient management recommendations;
computer readable code for storing, displaying and communicating said patient management recommendations.

46. A computer program according to claim 45 further comprising computer readable code for communicating said patient-specific data and medication specific data from at least one remote location to a central location for analysis and/or clinician evaluation.

47. A computer program according to claim 45 further comprising computer readable code for transmitting data and analysis across the internet.

48. A computer program according to claim 45 further comprising computer readable code that analyzes said patient management rules, patient management recommendations, and inputs and identifies inputs, analyses or recommendations that require clinician review and communicates said inputs, analyses or recommendations to a clinician.

49. A computer program according to claim 45 wherein a clinician can modify said patient management rules.

50. A computer program according to claim 45 wherein said anticoagulant data further includes at least one element from the group comprising: commercially available dosages, contraindications, influencing variables, pharmacology, adverse effects, pharmacokinetic parameters and recommended monitoring frequency.

51. A computer program according to claim 45 wherein said patient-specific data further includes at least on element from the group comprising medical status, evidence of bleeding, diet, exercise, alcohol consumption, changes in international normalized ratio data, consumption of foods high in vitamin K, use of vitamin K, use of herbal supplement, patient medication compliance, patient pharmacokinetic parameters, use of supplements or vitamins, weight and age.

52. A computer program of claim 45 further comprising a computer readable code for a scheduling module that provides a means for scheduling patient visits consistent with patient management recommendations.

53. A computer program of claim 45 that further comprises computer readable code that provides a means of receiving, storing and transmitting patient education material, selecting said patient education material based on said patient management recommendations, and printing said patient educational material.

54. A computer program of claim 45 further comprising an algorithm for computing recommended dosages for at least one anticoagulant based on accepted standardized ranges.
